(19) United States
(12) Patent Application Publication
(10) Pub. No.: US 2005/0095703 A1
(43) Pub. Date: May 5, 2005

(54) Method for the establishment of a pluripotent human blastocyst - derived stem cell line

(76) Inventors: Henrik Semb, Bjarred (SE); Anna Tonning, Goteborg (SE)

Correspondence Address: BURNS DOANE SWECKER & MATHIS L L P, POST OFFICE BOX 1404, ALEXANDRIA, VA, 22313-1404, US

(21) Appl. No.: 10/500,118

(22) PCT Filed: Dec. 27, 2002
(86) PCT No.: PCT/EP02/14895

Related U.S. Application Data
(60) Provisional application No. 60/342,771, filed on Dec. 28, 2001.

(30) Foreign Application Priority Data
Dec. 28, 2001 (SE) 0104471-8

(51) Int. Cl. C12N 5/08

(52) U.S. Cl. 435/366

(57) ABSTRACT

The present invention concerns a method for the establishment of pluripotent human blastocyst-derived stem (BS) cell lines, stem cells obtained by the method, differentiation of these cells into differentiated cells, the differentiated cells, and the use of these differentiated cells in the preparation of medicaments. The undifferentiated pluripotent stem cells can be made to differentiate to a number of specialized cell types which can be utilized in the manufacture of medicaments for treating a number of conditions or pathologies involving degeneration of tissue, e.g., of the pancreas leading to, e.g., development of diabetes, or of the CNS (e.g., Alzheimer's, Parkinson's disease, etc.) or degeneration of the CNS caused by e.g., stroke or physical trauma.

### FIELD OF THE INVENTION

The present invention concerns a method for the establishment of a pluripotent human blastocyst-derived stem (BS) cell line, stem cells obtained by the method, differentiation of these cells into differentiated cells, the differentiated cells and the use of these differentiated cells in the preparation of medicaments. The undifferentiated pluripotent stem cells can be made to differentiate to a number of specialized cell types which can be utilized in the manufacture of medicaments for treating a number of conditions or pathologies involving degeneration of tissue e.g. of the pancreas leading to e.g. development of diabetes, or of the CNS (e.g. Alzheimer's, Parkinson's disease etc.) or degeneration of the CNS caused by e.g. stroke or physical trauma.

### BACKGROUND OF THE INVENTION

A stem cell is a cell type that has a unique capacity to renew itself and to give rise to specialized or differentiated cells. Although most cells of the body, such as heart cells or skin cells, are committed to conduct a specific function, a stem cell is uncommitted, until it receives a signal to develop into a specialized cell type. What makes the stem cells unique is their proliferative capacity, combined with their ability to become specialized. For years, researchers have focused on finding ways to use stem cells to replace cells and tissues that are damaged or diseased. So far, most research has focused on two types of stem cells, embryonic and somatic stem cells. Embryonic stem cells are derived from the pre-implanted fertilized oocyte, i.e. blastocyst, whereas the somatic stem cells are present in the adult organism, e.g. within the bone marrow, epidermis and intestine. Pluripotency tests have shown that whereas the embryonic or blastocyst-derived stem cells (hereafter referred to as blastocyst-derived stem cells or BS cells) can give rise to all cells in the organism, including the germ cells, somatic stem cells have a more limited repertoire in descendent cell types.

In 1998, investigators were for the first time able to isolate BS cells from human fertilized oocytes and to grow them in culture see e.g. U.S. Pat. No. 5,843,780 and in U.S. Pat. No. 6,200,806.

The procedure used in the patent specifications mentioned above depends on the use of blastocysts with an intact zona pellucida. Furthermore, the method disclosed in these patents specifically use inner cell mass cells that have been isolated by immunosurgery for plating on mouse embryonic feeder cells. This method has several drawbacks, for example, it is time consuming, technically difficult and results in low yields of stem cells. Taken together, these drawbacks make it a costly method.

So far, only two articles have been published on establishment and characterization of hBS cells. This low number illustrates the unexpected problems associated with establishing these stem cells from human blastocysts. As a result very few hBS cell lines are available. The present invention describes a method for the preparation of hBS cell lines and a combination of method steps that independently will not be sufficient for deriving hBS cells but when used together they constitute the minimal requirement for successful derivation of hBS cells.

Furthermore, the present invention allows a successful derivation of hBS stem cell lines from hatched and intact blastocysts and allows for derivation of hBS cell lines after plating blastocysts onto feeder cells.

One of the difficulties with previously described methods has been to achieve an efficient attachment of the blastocysts to the feeder cells. This has resulted in low yields of end-product cells. The present invention addresses this problem.

Perhaps the most far-reaching potential application of hBS cells is the generation of cells and tissue that could be used for so-called cell therapies. Many diseases and disorders result from disruption of cellular function or destruction of tissues of the body. Today, donated organs and tissues are often used to replace ailing or destroyed tissue. Unfortunately, the number of people suffering from disorders suitable for treatment by these methods far outstrips the number of organs available for transplantation. The availability of hBS cells and the intense research on developing efficient methods for guiding these cells towards different cell fates, e.g. insulin-producing β-cells, cardiomyocytes, and dopamine-producing neurons, holds growing promise for future applications in cell-based treatment of degenerative diseases, such as diabetes, myocardial infarction and Parkinson's.

### DESCRIPTION OF THE INVENTION

The inventors have established a novel method for establishing a pluripotent human blastocyst-derived stem cell line from a fertilized oocyte, including propagation of the cell line in an undifferentiated state.

Thus, the present invention relates to a method for obtaining a pluripotent human blastocyst-derived stem cell line, the method comprising the steps of
i) using a fertilized oocyte optionally, having a grade 1 or 2, to obtain a blastocyst, optionally having a grade A or B,
ii) co-culturing the blastocyst with feeder cells for establishing one or more colonies of inner cell mass cells,
iii) isolating the inner cell mass cells by mechanical dissection,
iv) co-culturing of the inner cell mass cells with feeder cells to obtain a blastocyst-derived stem cell line.
v) optionally, propagation of the blastocyst-derived stem cell line.

In accordance with to the above, it is one object of the present invention to provide a method for establishing an undifferentiated human blastocyst-derived stem cell line. As a starting material for this procedure, fertilized oocytes are used. The quality of the fertilized oocytes is of importance for the quality of the resulting blastocysts.

In the method of the present invention, the establishment and evaluation of blastocysts are performed as described below. The human blastocysts in step i) of the method may be derived from frozen or fresh human in vitro fertilized oocytes. In the following is described a procedure for selecting suitable oocytes for use in a method according to the present invention. The present inventors have found that an important success criterion for the present method is a proper selection of oocytes. Thus, if only grade 3 oocytes are applied, the probability of obtaining a hBS cell line fulfilling the general requirements (described below) is low.

Donated fresh fertilized oocytes: On day 0 the oocyte is aspirated in Asp-100 (Vitrolife), and fertilized on day 1 in IVF-50 (Vitrolife). The fertilized oocyte is evaluated based on morphology and cell division on day 3. The following scale is used for fertilized oocyte evaluation:
Grade 1 fertilized oocyte: Even blastomers, no fragments
Grade 2 fertilized oocyte: <20% fragments
Grade 3 fertilized oocyte: >20% fragments

After evaluation on day 3, fertilized oocytes of grade 1 and 2 are either implanted or frozen for storage. Fertilized oocytes of grade 3 are transferred to ICM-2 (Vitrolife). The fertilized oocytes are further cultured for 3-5 days (i.e. day 5-7 after fertilization). The blastocysts are evaluated according to the following scale:
Grade A Blastocyst: Expanded with distinct inner cell mass (ICM) on day 6
Grade B Blastocyst: Not expanded but otherwise like grade A
Grade C Blastocyst: No visible ICM

Donated frozen fertilized oocytes: At day 2 (after fertilization) the fertilized oocytes are frozen at the 4-cell stadium using Freeze-Kit (Vitrolife). Frozen fertilized oocytes are stored in liquid nitrogen. Informed consent is obtained from the donors before the 5-year limit has passed. The fertilized oocytes are thawed using Thaw-Kit (Vitrolife), and the procedure described above is followed from day 2.

As described above, fresh fertilized oocytes are from grade 3 quality, and frozen fertilized oocytes are from grade 1 and 2. According to data obtained by the methods of the present invention, the percentage of fresh fertilized oocytes that develop into blastocysts is 19%, while 50% of the frozed fertilized oocytes develop into blastocysts. This means that the frozen fertilized oocytes are much better for obtaining blastocysts, probably due to the higher quality of the fertilized oocytes. 11% of the blastocysts derived from fresh fertilized oocytes develop into a stem cell line, while 15% of the blastocysts derived from frozen fertilized oocytes develop into a stem cell line. In summary, of the fertilized oocytes that were put into culture 2% of fresh fertilized oocytes developed into a stem cell line, and 7% of frozen fertilized oocytes that were put into culture developed into a stem cell line.

The culturing of the fertilized oocyte to the blastocyst-stage is performed after procedures well-known in the art. Procedures for preparing blastocysts may be found in Gardner et al, Embryo culture systems, In Trounson, A. O., and Gardner, D. K. (eds), Handbook of in vitro fertilization, second edition. CRC Press, Boca Raton, pp. 205-264; Gardner et al, Fertil Steril, 74, Suppl 3, O-086; Gardner et al, Hum Reprod, 13, 3434,3440; Gardner et al, J Reprod Immunol, In press; and Hooper et al, Biol Reprod, 62, Suppl 1, 249.

After establishment of blastocysts in step i) optionally derived from fertilized oocytes having grade 1 or 2, the blastocysts having grade A or B are co-cultured with feeder cells for establishing one or more colonies of inner cell mass cells. After being plated onto feeder cells, their growth is monitored and when the colony is large enough for manual passaging (approximately 1-2 weeks after plating), the cells may be dissected from other cell types and expanded by growth on new feeder cells. The isolation of the inner cell mass cells is performed by mechanical dissection, which may be performed by using glass capillaries as a cutting tool. The detection of the inner cell mass cells is easily performed visually by microscopy and, according, it is not necessary to use any treatment of the oocytes with enzymes and/or antibodies to impair or remove the trophectoderm.

Thus, the procedure alleviates the need for immunosurgery. By comparing the success-rate in using immunosurgery versus the present method, which leaves the trophectoderm intact, it has been observed that the much simpler, faster and non-traumatic procedure of avoiding immunosurgery is more efficient than immunosurgery. The novel procedures make the preparation of stem cell lines, and the differentiation of these cell lines commercially feasible. From a total of 122 blastocysts, 19 cell lines were established (15.5%). 42 blastocysts were processed by immunosurgery and 6 of these resulted in successfully established cell lines (14%). Eighty blastocysts were processed by the present method and 13 cell lines were established (16%).

Subsequent to dissection of the inner cell mass, the inner cell mass cells are co-cultured with feeder cells to obtain a blastocyst-derived stem (BS) cell line. After obtaining the BS cell line, the cell line is optionally propagated to expand the amount of cells. Thus, the present invention relates to a method as described above wherein the blastocyst-derived stem cell line is propagated. In one aspect, the invention relates to a method in which the propagation of blastocyst-derived stem cell line comprises passage of the stem cell line every 4-5 days. If the stem cell line is cultured longer than 4-5 days before passage, there is an increased probability that the cells undesirably will differentiate.

A specific procedure of passaging the cells is given in Example 5 herein.

Human BS cell lines may be isolated either from spontaneously hatched blastocysts or from expanded blastocysts with an intact zona pellucida. Thus the present invention relates to a method as described above in which the blastocyst in step i) is a spontaneously hatched blastocyst. For hatched blastocysts the trophectoderm may be left intact. Either hatched blastocysts or blastocysts with a removed or partially removed zona pellucida may be put on inactivated feeder cells.

Zona pellucida of the blastocyst may be at least partially digested or chemically frilled prior to step ii) e.g. by treatment with one or more acidic agents such as, e.g., ZD™-10 (Vitrolife, Gothenburg, Sweden), one or more enzymes or mixture of enzymes such as pronase.

A brief pronase (Sigma) treatment of blastocysts with an intact zona pellucida results in the removal of the zona. Other types of proteases with the same or similar protease activity as pronase may also be used. The blastocysts can be plated onto said inactivated feeder cells following the pronase treatment.

In an embodiment of the invention step ii) and/or step iv) may be performed in an agent that improves the attachment of the blastocysts and/or if relevant the inner cell mass cells to the feeder cells.

A suitable substance for this purpose is a hyaluronic acid.

A suitable medium for plating the blastocysts onto feeder cells can be BS-medium that may be complemented with hyaluronic acid, which seems to promote the attachment of the blastocysts on the feeder cells and growth of the inner cell mass. Hyaluronan (HA) is an important glycosaminoglycan constituent of the extracellular matrix in joints. It appears to exert its biological effects through binding interactions with at least two cell surface receptors: CD44 and receptor for HA-mediated motility (RHAMM), and to proteins in the extracellular matrix. The positive effects of HA during the establishment of hBS cells may be exerted through its interactions with the surfactant polar heads of phospholipids in the cell membrane, to thereby stabilize the surfactant layer and thus lower the surface tension of the inner cell mass or blastocyst which may result in increased efficiency in binding to the feeder cells. Alternatively, HA may bind to its receptors on the inner cell mass or blastocyst and/or to the feeder cells and exert biological effects which positively influence the attachment and growth of the inner cell mass. According to this, other agents that may alter the surface tension of fluids, or in other ways influence the interaction between the blastocyst and feeder cells can also be used in instead of hyaluronic acid.

The inventors have also found that the culturing of the feeder cells is of importance for the establishment of the hBS cell line. In one embodiment, the propagation of blastocyst-derived stem cell line comprises passage of the feeder cells at the most 3 times, such as e.g. at the most 2 times.

Suitable feeder cells for use in a method of the invention are embryonic feeder cells. In a method according to the invention the feeder cells employed in steps ii) and iv) are the same or different and originate from animal source such as e.g. any mammal including human, mouse, rat, monkey, hamster, frog, rabbit etc. Feeder cells from human or mouse species are preferred.

Another important criterion for obtaining an hBS cell line fulfilling the general requirements are the conditions under which the blastocysts are cultured. The blastocyst-derived stem cell line may accordingly by propagated by culturing the stem cells with feeder cells of a density of less than about 60,000 cells per cm², such as e.g. less than about 55,000 cells per cm², or less than about 50,000 cells per cm². In a specific embodiment, the propagation of blastocyst-derived stem cell line comprises culturing the stem cells with feeder cells of a density of about 45,000 cells per cm². These values apply in those cases where mouse feeder cells are used and it is contemplated that a suitable density can be found for other types of feeder cells as well. Based on the findings of the present inventors, a person skilled in the art will be able to find such suitable densities.

In a method according to the invention, the feeder cells may be mitotically inactivated in order to avoid unwanted growth of the feeder cells.

The blastocyst-derived stem cell line obtained by the present invention maintains selfrenewal and pluripotency for a suitable period of time and, accordingly it is stable for a suitable period of time. In the present context the term “stable” is intended to denote proliferation capacity in an undifferentiated state for more than 21 months when grown on mitotically inactivated embryonic feeder cells.

The stem cell line obtained by the present invention fulfils the general requirements. Thus, the cell line
i) exhibits proliferation capacity in an undifferentiated state for more than 21 months when grown on mitotically inactivated embryonic feeder cells, and
ii) exhibits normal euploid chromosomal karyotype, and
iii) maintains potential to develop into derivatives of all types of germ layers both in vitro and in vivo, and
iv) exhibits at least two of the following molecular markers OCT-4, alkaline phosphatase, the carbohydrate epitopes SSEA-3, SSEA4, TRA 1-60, TRA 1-81, and the protein core of a keratin sulfate/chondroitin sulfate pericellular matrix proteinglycan recognized by the monoclonal antibody GCTM-2, and
v) does not exhibit molecular marker SSEA-1 or other differentiation markers, and
vi) retains its pluripotency and forms teratomas in vivo when injected into immuno-compromised mice, and
vii) is capable of differentiating.

The undifferentiated hBS cells according to the present invention is defined by the following criteria; they were isolated from human pre-implantation fertilized oocytes, i.e. blastocysts, and exhibit a proliferation capacity in an undifferentiated state when grown on mitotically inactivated feeder cells; they exhibit a normal chromosomal karyotype; they express typical markers for undifferentiated hBS cells, e.g. OCT4, alkaline phosphatase, the carbohydrate epitopes SSEA-3, SSEA4, TRA 1-60, TRA 1-81, and the protein core of a keratin sulfate/chondroitin sulfate pericellular matrix proteinglycan recognized by the monoclonal antibody GCTM-2, and do not show any expression of the carbohydrate epitope SSEA-1 or other differentiation markers. Furthermore, pluripotency tests in vitro and in vivo (teratomas) demonstrate differentiation into derivatives of all germ layers.

According to the above, the invention is an essentially pure preparation of pluripotent human BS cells, which i) exhibits proliferation capacity in an undifferentiated state for more than 21 months when grown on mitotically inactivated embryonic feeder cells; ii) exhibits normal euploid chromosomal karyotype; iii) maintains potential to develop into derivatives of all types of germ layers both in vitro and in vivo; iv) exhibits at least two of the following molecular markers OCT-4, alkaline phosphatase, the carbohydrate epitopes SSEA-3, SSEA4, TRA 1-60, TRA 1-81, and the protein core of a keratin sulfate/chondroitin sulfate pericellular matrix proteinglycan recognized by the monoclonal antibody GCTM-2 v) does not exhibit molecular marker SSEA-1 or other differentiation markers, and vi) retains its pluripotency and forms teratomas in vivo when injected into immuno-compromised mice, and vii) is capable of differentiating.

Procedures for the detection of cell markers can be found in Gage, F. H., Science, 287:1433-1438 (2000). These procedures are well known for the skilled person and include methods such as RT-PCR or immunological assays where antibodies directed against the cell markers are used. In the following, methods for detection of cell markers, hybridisation methods, karyotyping, methods for measuring telomerase activity and teratoma formation are described. These methods can be used to investigate whether the hBS cells obtained according to the present invention fulfil the above-mentioned criteria.

Immunohistochemistry

The human BDP stem cells maintained in culture are routinely monitored regarding their state of differentiation. Cell surface markers used for monitoring the undifferentiated BS cells are SSEA-1, SSEA-3, SSEA-4, TRA-1-60, TRA-1-81. Human BDP stem cells are fixed in 4% PFA and subsequently permeabilized using 0.5% Triton X-100. After washing and blocking with 10% dry milk the cells are incubated with the primary antibody. After extensive washes the cell are incubated with the secondary antibody and the nuclei are visualized by DAPI staining.

Alkaline Phosphatase

The activity of alkaline phosphatase is determined using a commercial available kit following the instructions from the manufacturer (Sigma Diagnostics).

Oct-4 RT-PCR

The mRNA levels for the transcription factor Octet is measured using RT-PCR and gene specific primer sets (5′-CGTGMGCTGGAGMGGAGAAGCTG, 5′-CMGGGCCGCAGCTTACACATGTTC) and GAPDH as housekeeping gene (5′-ACCACAGTCCATGCCATCAC, 5′-TCCACCACCCTGTTGCTGTA).

Fluorescence In Situ Hybridization (FISH)

In one round of FISH one ore more chromosomes are being selected with chromosome specific probes. This technique allows numerical genetic aberrations to be detected, if present. For this analysis CTS uses a commercially available kit containing probes for chromosome 13, 18, 21 and the sex chromosomes (X and Y) (Vysis. Inc, Downers Grove, Ill., USA). For each cell line at least 200 nuclei are being analyzed. The cells are resuspended in Camoy's fixative and dropped on positively charged glass slides. Probe LSI 13/21 is mix with LSI hybridization buffer and added to the slide and covered with a cover slip. Probe CEP X/Y/18 is mixed with CEP hybridization buffer and added in the same way to another slide. Denaturing is performed at 70° C. for 5 min followed by hybridization at 37° C. in a moist chamber for 14-20 h. Following a three step washing procedure the nuclei are stained with DAPI II and the slides analyzed in an invert microscope equipped with appropriate filters and software (CytoVision, Applied Imaging).

Karyotyping

Karyotyping allows all chromosomes to be studied in a direct way and is very informative, both numerical and larger structural aberrations can be detected. In order to detect mosaicism, at least 30 karyotypes are needed. However, this technique is both very time consuming and technically intricate. To improve the conditions for the assay the mitotic index can be raised by colcemid, a synthetic analog to colchicin and a microtubule-destabilizing agent causing the cell to arrest in metaphase, but still a large supply of cells are needed (6×10⁶ cells/analysis). The cells are incubated in the presence of 0.1 μg/ml colcemid for 1-2 h, and then washed with PBS and trypsinized. The cells are collected by centrifugation at 1500 rpm for 10 min. The cells are fixed using ethanol and glacial acetic acid and the chromosomes are visualized by using a modified Wrights staining.

Comparative Genomic Hybridization

Comparative genomic hybridization (CGH) is complementary to karyotyping. CGH gives a higher resolution of the chromosomes and is technically less challenging. Isolated DNA is nicktranslated in a mixture of DNA, A4, Texas red-dUTP/FITC 12-dUTP, and DNA polymerase I. An agarose gel electrophoresis is performed to control the size of resulting DNA fragments (600-2000 bp). Test and reference DNA is precipitated and resuspended in hybridization mixture containing formamide, dextrane sulfate and SSC. Hybridization is performed on denatured glass slides with metaphases for 3 days at 37° C. in a moist chamber. After extensive washing one drop of antifade mounting mixture (vectashield, 0,1 μg/ml DAPI II) is added and the slides covered with cover slips. Slides are subsequently evaluated under a microscope and using an image analysis system.

Telomerase Activity

Since a high activity has been defined as a criterion for BS cells 6 the telomerase activity is measured in the BS cell lines. It is known that telomerase activity successively decrease when the cell reaches a more differentiated state. Quantifying the activity must therefore be related to earlier passages and control samples, and can be used as a tool for detecting differentiation. The method, Telomerase PCR ELISA kit (Roche) uses the internal activity of telomerase, amplifying the product by polymerase chain reaction (PCR) and detecting it with an enzyme linked immunosorbent assay (ELISA). The assay is performed according to the manufacturer's instructions. The results from this assay shows typically a high telomerase activity (>1) for BS cells.

The cell lines retain their pluripotency and forms teratomas in vivo when injected into immuno-compromised mice. In addition, in vitro these cells can form BS cell derived bodies. In both of these models, cells characteristic for all germ layers can be found.

Teratoma Formation in Immunodeficient Mice

One method to analyze if a human BS cell line has remained pluripotent is to xenograft the cells to immunodeficient mice in order to obtain tumors, teratomas. Various types of tissues found in the tumor should represent all three germlayers. Reports have showed various tissues in tumors derived from xenografted immunodeficient mice, such as striated muscle, cartilage and bone (mesoderm) gut (endoderm), and neural rosettes (ectoderm). Also, large portions of the tumors consist of disorganized tissue.

Severe combined immunodeficient (SCID)-mice, a strain that lack B- and T-lymphocytes are used for analysis of teratoma formation. Human BS cells are surgically placed in either testis or under the kidney capsule. In testis or kidney, BS cells are transplanted in the range of 10 000-100 000 cells. Ideally, 5-6 mice are used for each cell line at a time. Preliminary results show that female mice are more post-operative stable than male mice and that xenografting into kidney is as effective in generating tumors as in testis. Thus, a female SCID-mouse teratoma model is preferable. Tumors are usually palpable after approximate 1 month. The mice are sacrificed after 14 months and tumors are dissected and fixed for either paraffin-or freeze-sectioning. The tumor tissue is subsequently analyzed by immunohistochemical methods. Specific markers for all three germlayers are used. The markers currently used are: human E-Cadherin for distinction between mouse tissue and human tumour tissue, α-smooth muscle actin (mesoderm), α-Fetoprotein (endoderm), and β-III-Tubulin (ectoderm). Additionally, hematoxylin-eosin staining is performed for general morphology.

The hBS cell line obtained by the method according to the method of the present invention can be used for the preparation of differentiated cells. Therefore the invention also relates to such differentiated cells.

In a further embodiment, the hBS cell line according to the invention has the ability of differentiating into an insulin producing cells. They may be capable of forming islet-like structures, and the amount of insulin producing β-cells is generally higher than 25%, such as e.g. higher than 35%, or higher than 40%, or higher than 45%, or higher than 50%.

Thus in one embodiment, the insulin producing cells produces at least about 300 ng insulin/mg total protein such as at least about 380 ng insulin/mg total protein or at least about 450 ng insulin/mg total protein.

The blastocyst-derived stem cells may have the ability to differentiate into differentiated cells, which display the expression of pancreatic cell type markers, including at least one of insulin, Glut-2, Pdx-1, glucokinase, glucagon and somatostatin.

Alternatively the hBS cells have the ability to differentiate into insulin-producing cells characterized by their organization into islet-like structures comprising an inner core of β-cells surrounded by an outer layer of neuron-type cells, which neuron-type cells display expression of at least one of the following neuronal cell type markers, including neuron-specific β-III tubulin (TUJ1), NeuN, DoubleCortin, tyrosine hydroxylase and Map 2.

An object of the invention is also to provide an essentially pure preparation of BS stem cells that can be made to differentiate into oligodendrocytes, and also to provide an essentially pure preparation of oligodendrocytes prepared by this method. Oligodendrocytes can be characterized by the presence of cell markers such s RIP, GalC or O4.

The blastocyst-derived stem cells that are capable of being made into differentiated cells may display the expression of at least one of the following neuronal cell type markers, including neuron-specific β-III tubulin (TUJ1), NeuN, DoubleCortin, tyrosine hydroxylase and Map 2.

In a still further aspect, the invention relates to the use of a preparation of differentiated cells derived from the blastocyst-derived stem cells obtained by a method according to the invention for the manufacture of a medicament for the prevention or treatment of pathologies or diseases caused by tissue degeneration.

A further object of the invention is to provide cells that may be used for the preparation of a medicament for treating and/or preventing diseases that may be cured by “cell genesis”. By the term “cell genesis” is meant the generation of new cells such as neurons, oligodendrocytes, schwann cells, astroglial cells, all blood cells, chondrocytes, cardiomyocytes, oligodendroglia, astroglia, and/or different types of epithelium, endothelium, liver-, kidney-, bone-, connective tissue-, lung tissue-, exocrine and endocrine gland tissue-cells.

In an embodiment, the invention relates to the use of a preparation of differentiated cells derived from the blastocyst-derived stem cells obtained for the manufacture of a medicament for the prevention or treatment of pathologies or diseases in the pancreas such as diabetes including diabetes type I.

The differentiated cells derived from the blastocyst-derived stem cell line obtained may also be used for the manufacture of a medicament for the prevention or treatment of pathologies or diseases in the nervous system. Such diseases include multiple schlerosis, spinal chord injury, encephalopathies, Parkinson's disease, Huntingdon's disease, stroke, traumatic brain injuries, hypoxia induced brain injuries, ischemia induced brain injuries, hypoglycemic brain injuries, degenerative disorders of the nervous system, brain tumors and neuropathies in the peripheral nervous system.

In a still further embodiment, the invention relates to a kit for performing the method according to the invention. The kit comprises at least a first and a second component in separate compartments. The components comprise an agent that improves the attachment of the blastocysts, a digestive agent, BS-cell medium and/or feeder cells or mixtures thereof.

The kit may further comprise blastocysts with an intact zona pelludica or spontaneously hatched blastocysts.

In another aspect, the invention relates to a method for producing an essentially pure preparation of insulin-producing differentiated stem cells, comprising the steps of;
i) expanding human blastocyst-derived stem cells by growing these on an inactivated feeder cell layer in a suitable medium;
ii) generating blastocyst-derived stem cell bodies by dissociating colonies formed in step i) into smaller aggregates or individual cells, followed by transferring said aggregates or individual cells to non-adherent containers where they are incubated in a suitable medium;
iii) plating the blastocyst-derived stem cell bodies in containers in a suitable medium;
iv) selecting nestin-positive neural precursors in ITFSn medium;
v) expanding pancreatic endocrine progenitor cells in, N2-medium comprising B27 media complement and basic fibroblast growth factor;
vi) changing the medium to a basic fibroblast growth factor-free N2 medium.

The manual dissection may be performed by using glass capillaries as a cutting tool.

The human blastocyst-derived stem cells employed in the above-mentioned method are typically those obtained as described herein.

More specifically the medium used in step i) is human blastocyst-derived stem cell medium, the medium used in step ii) is blastocyst-derived stem cell body medium, and the medium used in step iii) is blastocyst-derived stem cell body medium.

Nicotinamide may be added after step vi).

A kit according to the invention may also be applied to the above-mentioned method. In this case, the kit comprises at least two of the following components in separate compartments; mitomycin C, hBS medium, BS cell body medium, ITSFn-medium, N2-medium, B27-media supplement, nicotinamide, and bFGF.

The kit may further comprise an essentially pure human blastocyst-derived stem cell line obtained by the method according to the present invention.

The invention is further illustrated by the following figures:

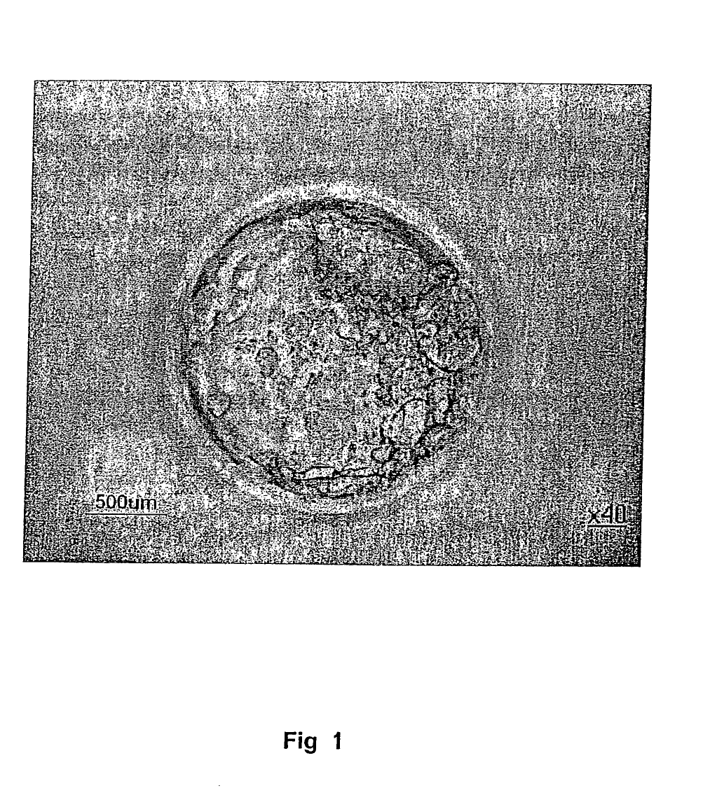

FIG. 1: Blastocyst (before pronase treatment) from which human BS cell line 167 was established.

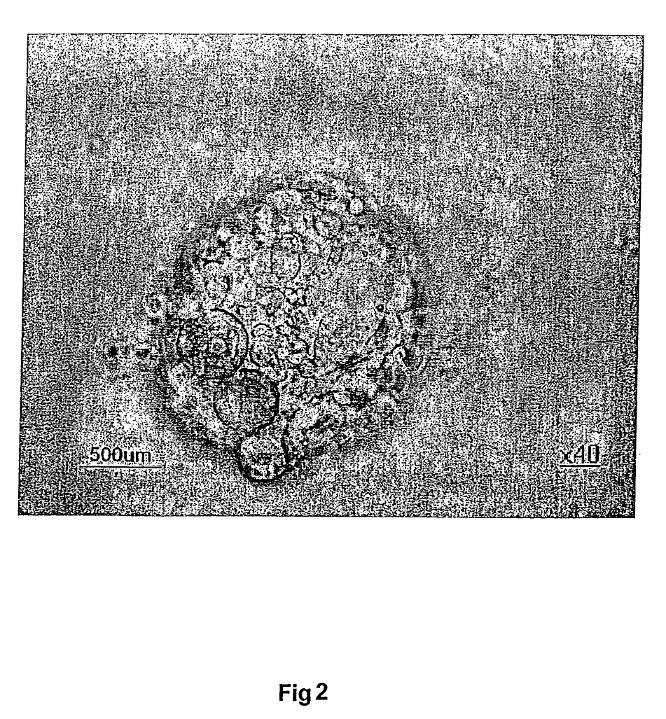

FIG. 2: Blastocyst (after pronase treatment) from which human BS cell line 167 was established.

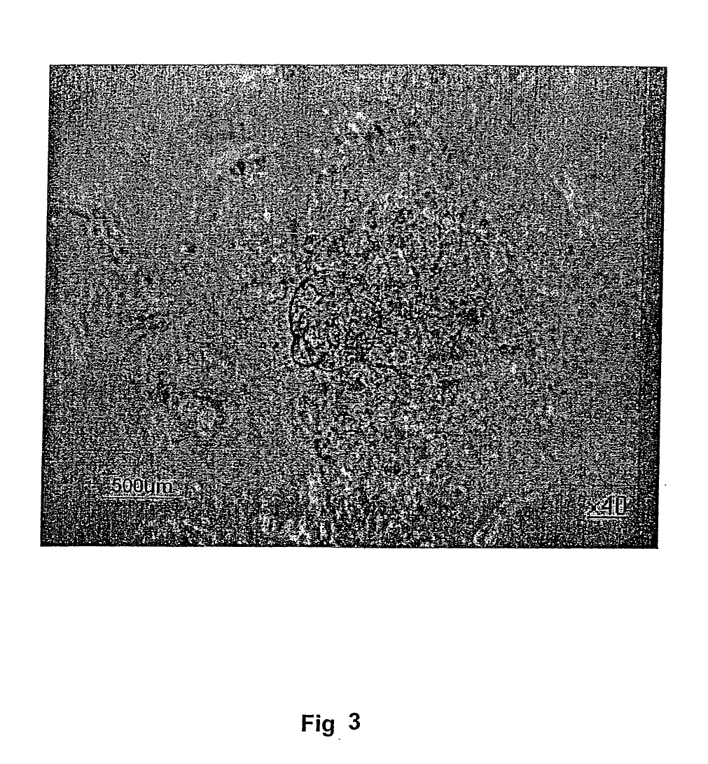

FIG. 3: Blastocyst 167 two days after plating on embryonic mouse fibroblasts.

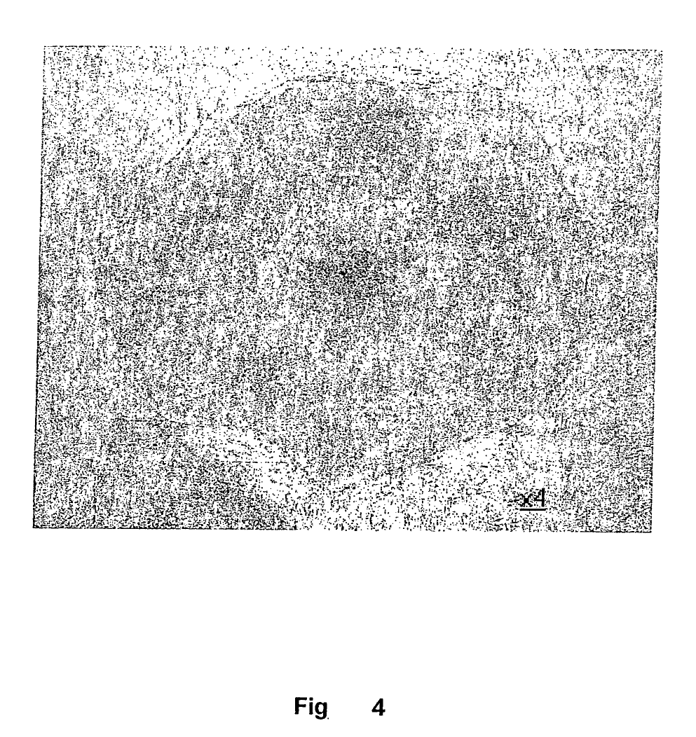

FIG. 4: Human BS cells at passage 69 cultured on embryonic mouse fibroblasts.

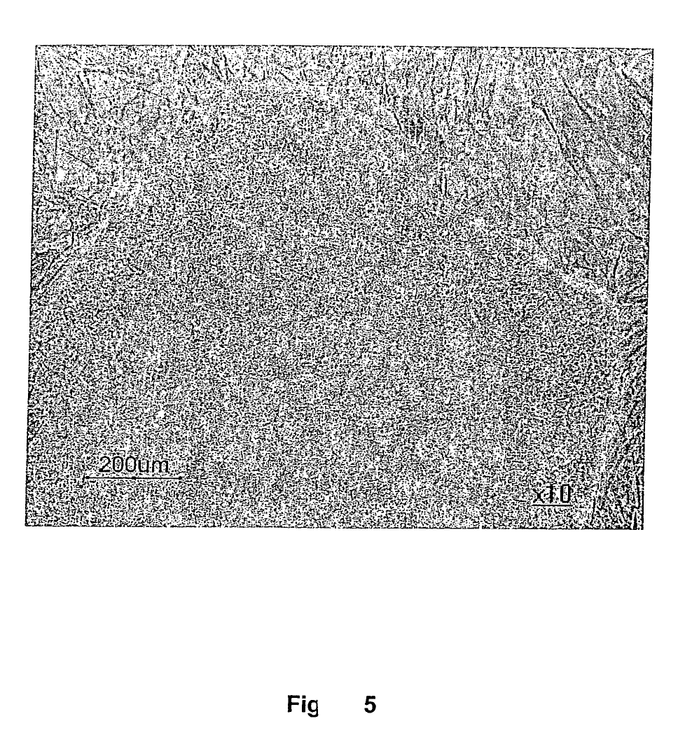

FIG. 5: Human BS cells at passage 71 cultured on embryonic mouse fibroblasts.

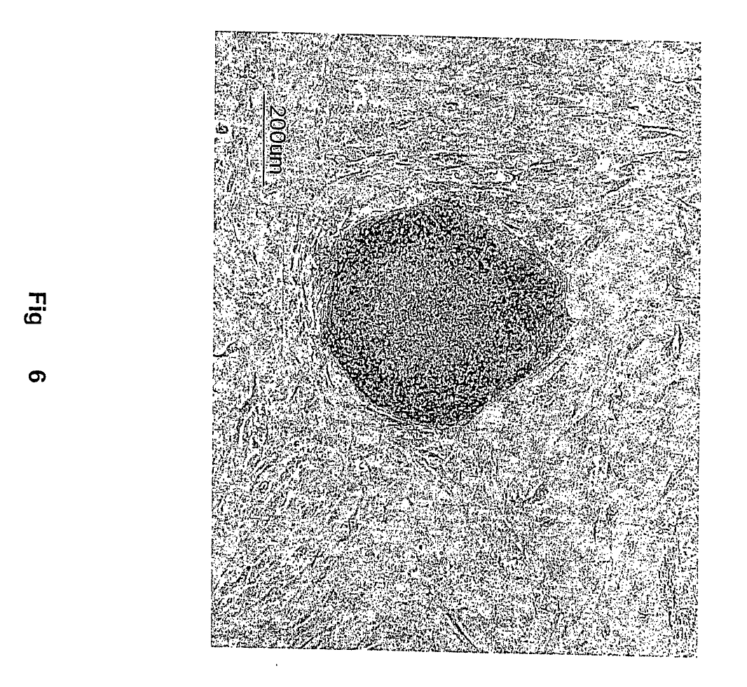

FIG. 6: Alkaline phosphatase in BS cells (10×)

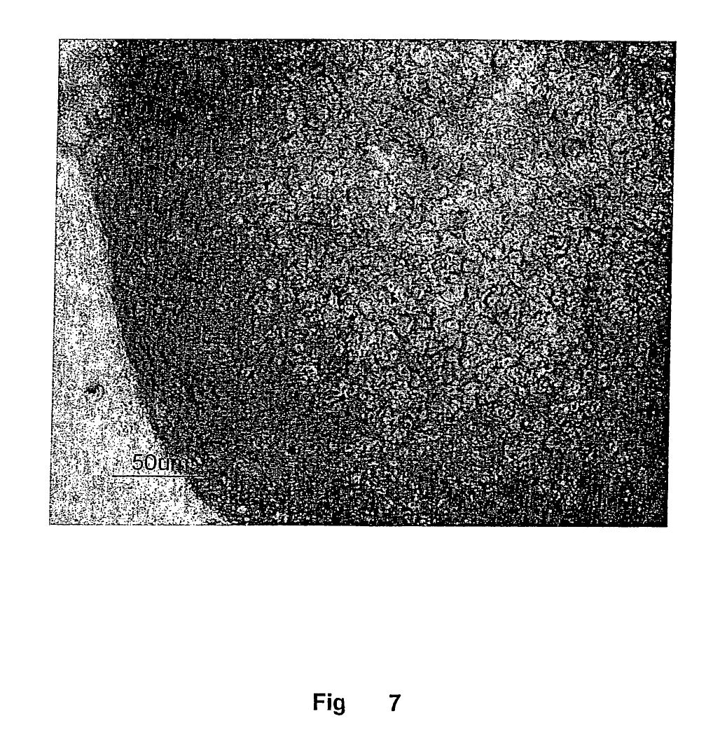

FIG. 7: Alkaline phosphatase in BS cells (40×)

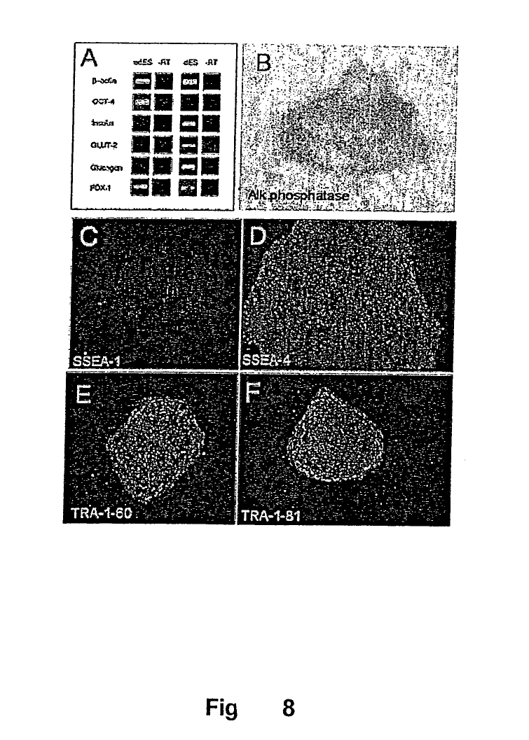

FIG. 8: Expression of molecular markers for undifferentiated human BS cells. (A) RT-PCR analysis of total RNA extracted from undifferentiated (ud) and from differentiated (d) human BS cells for the presence of Oct-4, insulin, GLUT-2, glucagon, and PDX-1 mRNA. In controls the reverse transcriptase was omitted (-RT). β-actin serves as housekeeping gene. (B) shows the presence of alkaline phosphatase by immunostaining in undifferentiated human BS cell colonies. (C) Analysis of SSEA-1 expression by immunostaining of undifferentiated human BS cell colonies. (D) Undifferentiated BS cells were immunopositive for SSEA-3 (data not shown) and SSEA-4. (E) Immunopositive human BS cell colonies for TRA-1-60 and in (F) for TRA-1-81 showing their undifferentiated status. Magnification 40×.

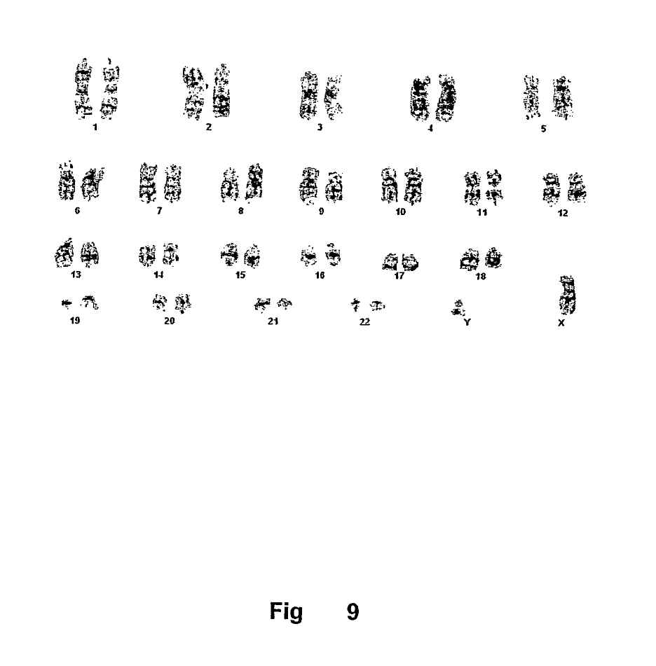

FIG. 9: Karyotyping of BS cells

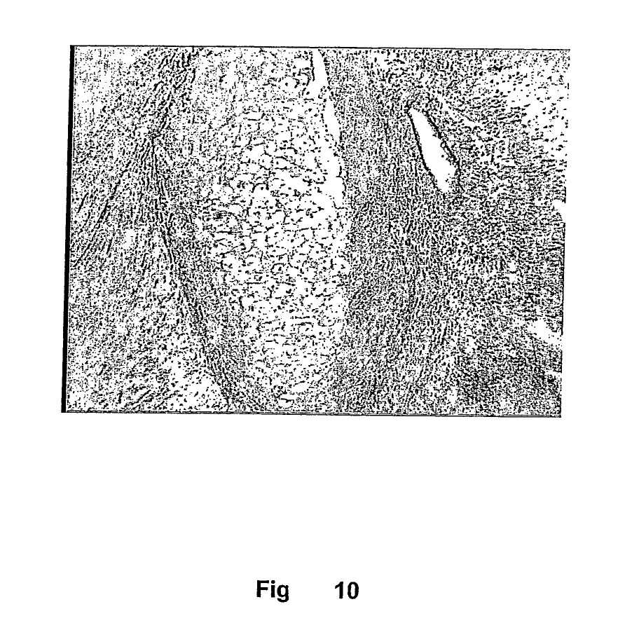

FIG. 10: Teratoma analysis: Bone

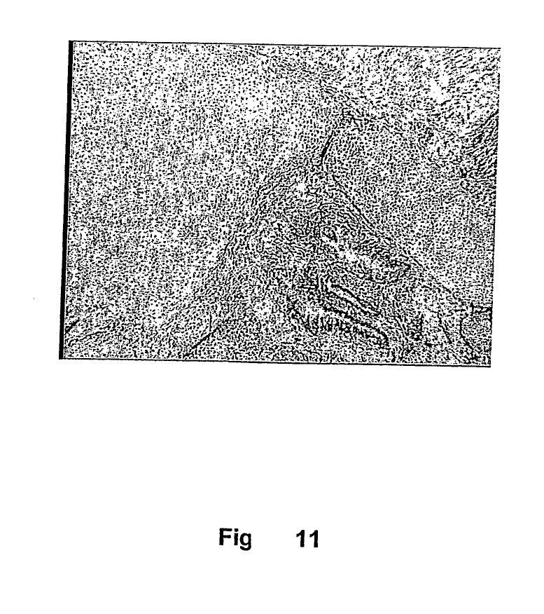

FIG. 11: Teratoma analysis: Cartilage

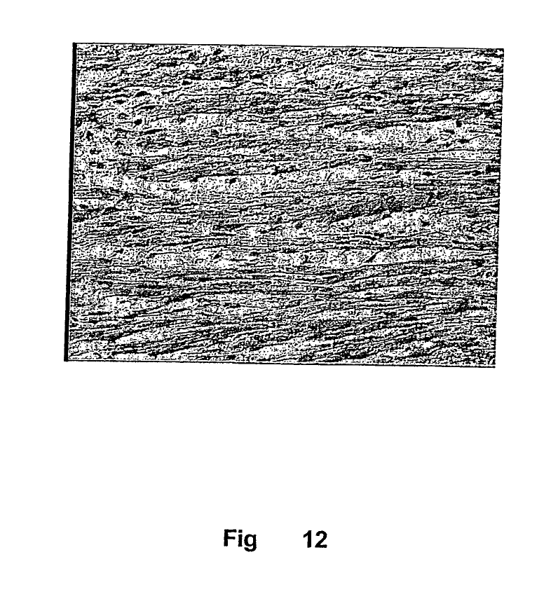

FIG. 12: Teratoma analysis: Skeletal muscle

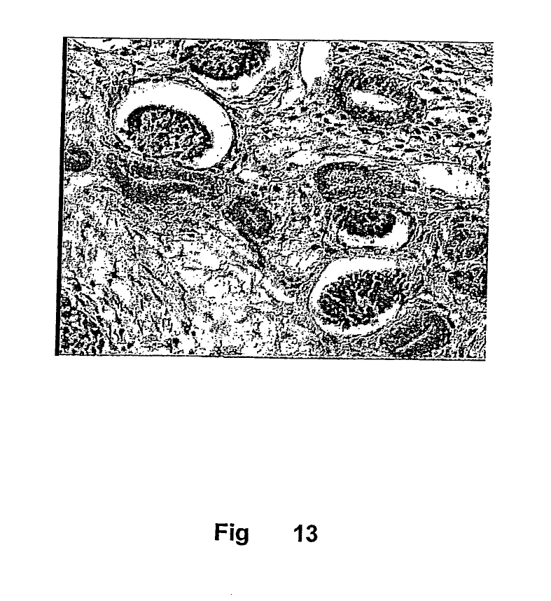

FIG. 13 Teratoma analysis: Kidney glomeruli

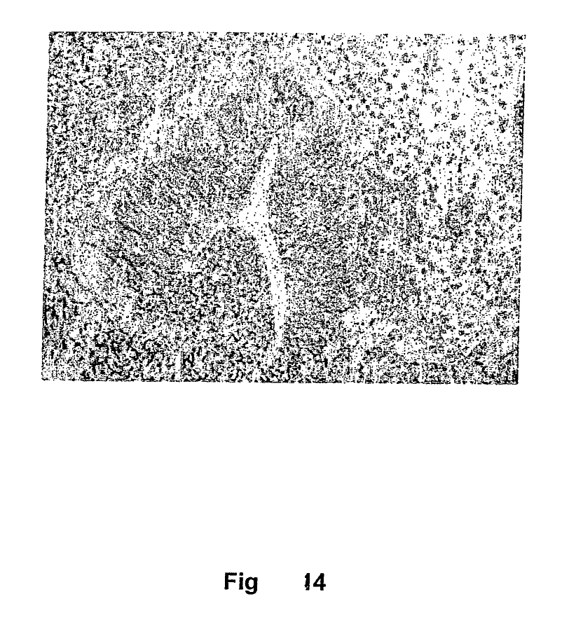

FIG. 14: Teratoma analysis: Rosettes of neural epithelium

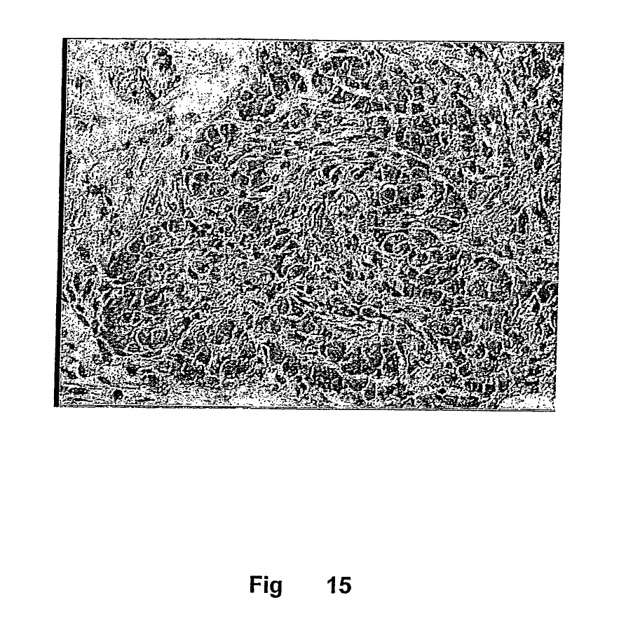

FIG. 15: Teratoma analysis: Glandular epithelium

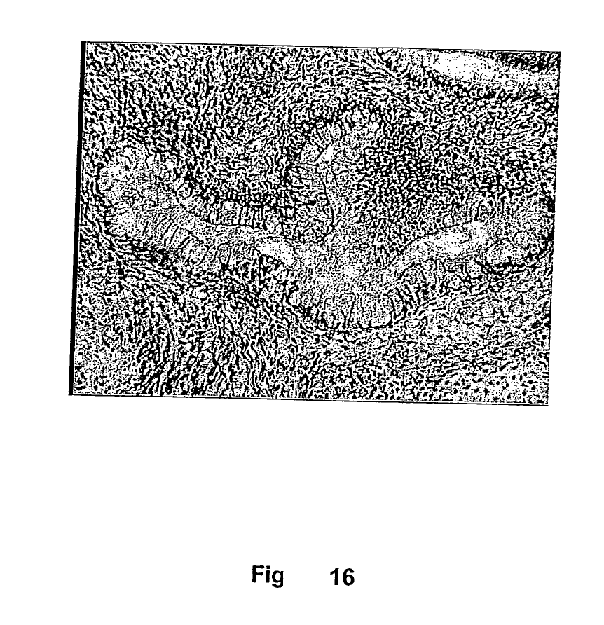

FIG. 16: Teratoma analysis: Mucous-producing epithelium

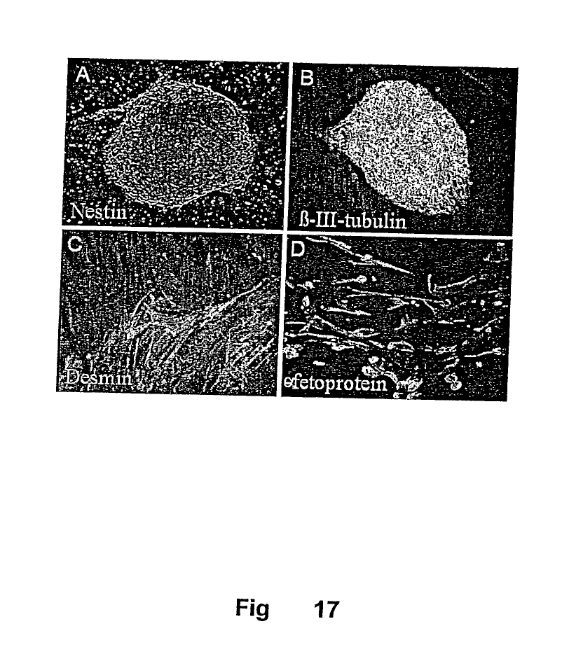

FIG. 17. Human BS cells differentiate in vitro into all germ layer cell types. Corresponding fluorescent micrographs show immunopositive cells stained with germ layer specific markers after 10 days in vitro. (A and B) show examples of neuroectodermal cells expressing nestin for neuronal precursors(A) and β-III-tubulin for postmitotic neurons (B) while (C) shows examples of mesodermal cells immunoreactive for Desmin; (D) examples of cells expressing α-fetoprotein.

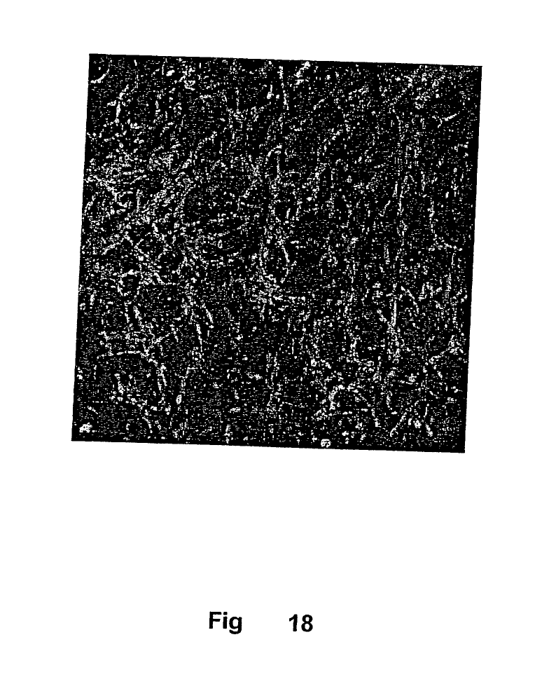

FIG. 18. Immuno staining for nestin in in vitro differentiated human BS cells.

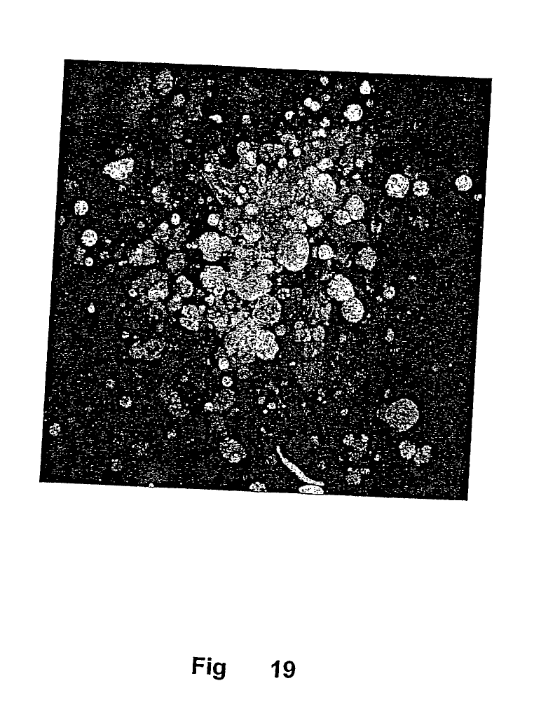

FIG. 19. Immuno staining for insulin in in vitro differentiated human BS cells.

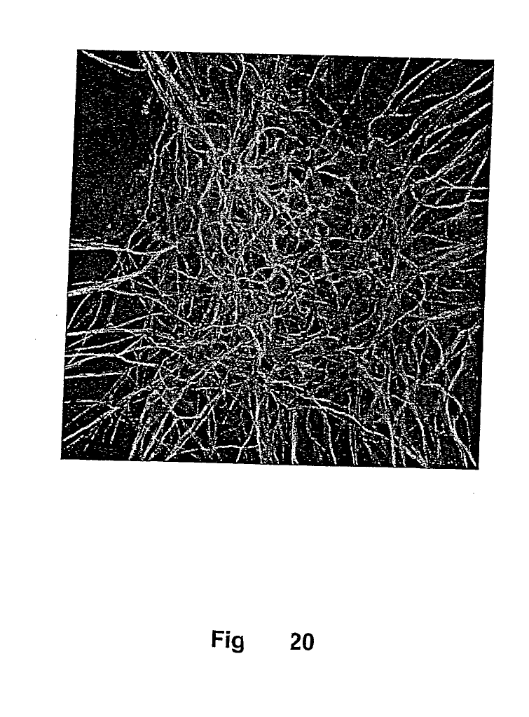

FIG. 20. Immuno staining for β-III-tubulin in in vitro differentiated human BS cells.

### DEFINITIONS AND ABBREVIATIONS

As used herein, the term “blastocyst-derived stem cell” is denoted BS cell, and the human form is termed “hBS cells”.

As used herein, the term “blastocyst-derived stem cell bodies” is denoted “BS cell bodies”.

As used herein, the term “EF cells” means “embryonic fibroblast feeder”. These cells could be derived from any mammal, such as mouse or human.

One suitable medium used in the invention is termed “BS-cell medium” or “BS-medium” and may be comprised of; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0,1 mM non-essential amino acids, 2 mM L-glutamine, 100 μM β-mercaptoethanol, 4 ng/ml human recombinant bFGF (basic fibroblast growth factor).

Another suitable medium for the present invention is “BS cell body medium”, this may be comprised as follows; KNOCKOUT® Dulbecco's Modified Eagle's Medium, supplemented with 20% KNOCKOUT® Serum replacement and the following constituents at their respective final concentrations: 50 units/ml penicillin, 50 pug/ml streptomycin, 0,1 mM nonessential amino acids, 2 mM L-glutamine and 100 μM β-mercaptoethanol (Itskovitz-Eldor, J. et al., 2000).

In the present context the term “stable” is intended to denote proliferation capacity in an undifferentiated state for more than 21 months when grown on mitotically inactivated embryonic feeder cells.

The invention will now be described with reference to the following examples. The examples are included herein for illustrative purposes only and are not intended to limit the scope of the invention in any way. The general methods described herein are well known to a person skilled in the art and all reagents and buffers are readily available, either commercially or easily prepared according to well-established protocols in the hands of a person skilled in the art. All incubations were in 37° C., under a CO₂ atmosphere.

### EXAMPLES

### Example 1

### Establishment of an Essentially Pure Preparation of Undifferentiated Stem Cells From Spontaneously Hatched Blastocysts

Human blastocysts were derived from frozen or fresh human in vitro fertilized embryos. Spontaneously hatched blastocysts were put directly on feeder cells (EF) in BS cell medium (KNOCKOUT Dulbecco's Modified Eagle's Medium, supplemented with 20% KNOCKOUT Serum replacement, and the following constituents at the final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor), supplemented with 0.125 mg/ml hyaluronic acid. After plating the blastocysts on the EF cells, growth was monitored and when the colony was large enough for manual passaging approximately 1-2 weeks after plating) the inner cell mass cells were dissected from other cell types and expanded by growth on new EF cells.

### Example 2

### Establishment of an Essentially Pure Preparation of Undifferentiated Stem Cells From Blastocysts with an Intact Zona Pellucida

For blastocysts with an intact zona pellucida, a brief pronase (10 U/ml, Sigma) incubation in rS2 (ICM-2) medium (Vitrolife, Gothenburg, Sweden) was used to digest the zona, after which the blastocyst was put directly on the EF cell layer in BS medium supplemented with hyaluronic acid (0.125 mg/ml).

### Example 3

### Histo-Chemical Staining for Alkaline Phosphatase

The cells were harvested for RT-PCR and histological (alkaline phosphatase) and immunocytochemical analysis (see below).

RNA isolation and RT-PCR. Total cellular RNA was prepared using Rneasy Mini Kit (Qiagen) according to the manufacturer's recommendations. The cDNA synthesis was carried out using AMV First Strand cDNA Synthesis Kit for RT-PCR (Roche) and PCR using Platinum Taq DNA Polymerase (Invitrogen). Histochemical staining for alkaline phosphatase was carried out using commercially available kit (Sigma) following the manufacturer's recommendations.

### Example 4

### Preparation and Culturing of hBS Cell Line

Mouse embryonic fibroblasts feeder cells were cultivated on tissue culture dishes in EMFI-medium: DMEM (Dulbecco's Modified Eagle's Medium), supplemented with 10% FCS (Fetal Calf Serum), 0,1 μM β-mercaptoehanol, 50 units/ml penicillin, 50 μg/ml streptomycin and 2 mM L-glutamine (GibcoBRL). The feeder cells were mitotically inactivated with Mitomycin C (10 μg/ml, 3 hrs). Human BS cell-colonies were expanded by manual dissection onto inactivated mouse embryonic fibroblasts feeder cells.

Human BS cells were cultured on mitotically inactivated mouse embryonic fibroblasts feeder cells in tissue culture dishes with BS-cell medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0,1 mM non-essential amino acids, 2 mM L-glutamine, 100 μM β-mercaptoethanol, 4 ng/ml human recombinant bFGF (basic fibroblast growth factor). Seven days after passage the colonies were large enough to generate BS cell bodies.

BS cell colonies were cut with glass capillaries into 0.4×0.4 mm pieces and plated on non-adherent bacterial culture dishes containing BS cell body medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0,1 mM non-essential amino acids, 2 mM L-glutamine and 100 μM β-mercaptoethanol (Itskovitz-Eldor, J. et al., 2000). The BS cell bodies, including cystic BS cell bodies, formed over a 7-9-day period.

### Example 5

### Passage of hBS Cells

Before passage the hBS cells are photographed using a Nikon Eclipse TE2000-U inverted microscope (10× objective) and a DXM 1200 digital camera. Colonies are passaged every 4-5 days. The colonies are big enough to be passaged when they can be cut in pieces (0.1-0.3×0.1-0.3 mm). The first time the cells are passaged, they have grown for 1-2 weeks and can be cut in approximately four pieces.

The colonies are focused, one by one, in a stereo-microscope and cut in a checkered pattern according to the size above. Only the inner homogeneous structure is passaged. Each square of the colony is removed with the knife, aspirated into a capillary and placed on new feeder cells (with the maximum age of 4 days). 10-16 squares are placed evenly in every new IVF-dish. The dishes are left five to ten minutes so the cells can adhere to the new feeder and then placed in an incubator. The hBS medium is changed three times a week. If the colonies are passaged, medium is changed twice that particular week. Normally a “half change” is made, which means that only half the medium is aspirated and replaced with the equal amount of fresh, tempered medium. If necessary the entire volume of medium can be changed.

### Example 6

### Vitrification of hBS Cells

Colonies with the appropriate undifferentiated morphology from the cell line are cut as for passage. 100-200 ml liquid nitrogen is sterile filtered into a sufficient amount of cryotubes. Two solutions A and B are prepared (A: 800 μl Cryo PBS with 1 M Trehalose, 100 μl etylen glycole and 100 μl DMSO, B: 600 μl Cryo PBS with 1 M Trehalose, 200 μl etylen glycole and 200 μl DMSO) and the colonies are placed in A for 1 minute and in B for 25 seconds. Closed straws are used to store the frozen colonies. After the colonies have been transferred to a straw, it is immediately placed in a cryotube with sterile filtered nitrogen.

### Example 7

### Seeding of Embryonic Mouse Feeder (EMFi) Cells

The cells are inactivated with EMFi medium containing Mitomycin C by incubation at 37° C. for 3 hours. IVF-dishes are coated with gelatin. The medium is aspirated and the cells washed with PBS. PBS is replaced with trypsin to detach the cells. After incubation, the trypsin activity is stopped with EMFi medium. The cells are then collected by centrifugation, diluted 1:5 in EMFi medium, and counted in a Bürker chamber. The cells are diluted to a final concentration of 170K cells/ml EMFi medium. The gelatin in the IVF-dishes is replaced with 1 ml cell suspension and placed in an incubator. EMFi medium is changed the day after the seeding.

### REFERENCES

1. Itskovitz-Eldor, J. et al. Differentiation of human embryonic stem cells into embryoid bodies compromising the three embryonic germ layers. Mol Med 6, 88-95. (2000).
2. Rizzino, A. & Crowley, C. Growth and differentiation of embryonal carcinoma cell line F9 in defined media. Proc Natl Acad Sci USA 77, 457-61. (1980).
3. Lee, S. H., Lumelsky, N., Studer, L., Auerbach, J. M. & McKay, R. D. Efficient generation of midbrain and hindbrain neurons from mouse embryonic stem cells. Nat Biotechnol 18, 675-9. (2000).
4. Johe, K. K., Hazel, T. G., Muller, T., Dugich-Djordjevic, M. M. & McKay, R. D. Single factors direct the differentiation of stem cells from the fetal and adult central nervous system. Genes Dev 10, 3129-40. (1996).
5. Lumelsky, N. et al. Differentiation of embryonic stem cells to insulin-secreting structures similar to pancreatic islets. Science 292, 1389-94. (2001).
6. Brewer, G. J., Torricelli, J. R., Evege, E. K. & Price, P. J. Optimized survival of hippocampal neurons in B27-supplemented Neurobasal, a new serum-free medium combination. J Neurosci Res 35, 567-76. (1993).
7. Otonkoski, T., Beattie, G. M., Mally, M. I., Ricordi, C. & Hayek, A. Nicotinamide is a potent inducer of endocrine differentiation in cultured human fetal pancreatic cells. J Clin Invest 92, 1459-66. (1993).
8. Assady, S., Maor, G., Michal, A., Itskovitz-Eldkor, J., Skkorecki, K. L. & Tzukerman, M., Insulin Production by human Embryonic Stem Cells. Diabetes 50, 1691-1697, 2001.
9. Gardner et al, Embryo culture systems, In Trounson, A O., and Gardner, D. K. (eds), Handbook of in vitro fertilization, second edition. CRC Press, Boca Raton, pp. 205-264;
10. Gardner et al, Fertil Steril, 74, Suppl 3, O-086;
11. Gardner et al, Hum Reprod, 13, 3434,3440;
12. Gardner et al, J Reprod Immunol, In press;
13. Hooper et al, Biol Reprod, 62, Suppl 1, 249.
14. Gage, F. H., Science, 287:1433-1438 (2000).

1. A method for obtaining a pluripotent human blastocyst-derived stem cell line comprising:
i) using a fertilized oocyte of grade 1 or 2 to obtain a blastocyst of grade A or B;
ii) co-culturing the blastocyst with feeder cells to establish one or more colonies of inner cell mass cells;
iii) isolating the inner cell mass cells by mechanical dissection; and
iv) co-culturing the inner cell mass cells with feeder cells to obtain a blastocyst-derived stem cell line.

2. A method for obtaining a pluripotent human blastocyst-derived stem cell line comprising:
i) using a fertilized oocyte of grade 1 or 2 to obtain a blastocyst;
ii) co-culturing the blastocyst with feeder cells to establish one or more colonies of inner cell mass cells;
iii) isolating the inner cell mass cells by mechanical dissection;
iv) co-culturing the inner cell mass cells with feeder cells to obtain a blastocyst-derived stem cell line.

3. A method for obtaining a pluripotent human blastocyst-derived stem cell line comprising:
i) using a fertilized oocyte to obtain a blastocyst of grade A or B;
ii) co-culturing the blastocyst with feeder cells to establish one or more colonies of inner cell mass cells;
iii) isolating the inner cell mass cells by mechanical dissection; and
iv) co-culturing the inner cell mass cells with feeder cells to obtain a blastocyst-derived stem cell line.

4. A method for obtaining a pluripotent human blastocyst-derived stem cell line comprising:
i) using a fertilized oocyte optionally of, grade 1 or 2 to obtain a blastocyst of optionally grade A or B;
ii) co-culturing the blastocyst with feeder cells to establish for establishing one or more colonies of inner cell mass cells,
iii) isolating the inner cell mass cells by mechanical dissection,
iv) co-culturing the inner cell mass cells with feeder cells to obtain a blastocyst-derived stem cell line; and
v) propagation of the blastocyst-derived stem cell line culturing the stem cells with feeder cells of a density of less than about 60,000 cells per cm².

5. The method of claim 1, wherein the blastocyst in step i) is a spontaneously hatched blastocyst.

6. The method of claim 1, wherein the blastocyst-derived stem cell line is stable.

7. The method of claim 1, wherein the blastocyst-derived stem cell line is propagated.

8. The method of claim 7, wherein propagating the blastocyst-derived stem cell line comprises passaging the stem cell line every 4-5 days.

9. The method of claim 7, wherein propagating the blastocyst-derived stem cell line comprises culturing the stem cells with feeder cells of a density of less than about 60,000 cells per cm².

10. The method of claim 9, wherein propagating the blastocyst-derived stem cell line comprises culturing the stem cells with feeder cells of a density of about 45,000 cells per cm².

11. The method of claim 7, wherein the propagation of blastocyst-derived stem cell line comprises passage of the feeder cells at the most 3 times.

12. The method of claim 1, wherein the zona pellucida of the blastocyst has been at least partially digested prior to step ii).

13. The method of claim 12, wherein the zona pellucida of the blastocyst has been at least partially digested with a digestive agent selected from the group comprising acidic reacting substances, enzymes and mixtures thereof.

14. The method of claim 1, wherein step ii) and/or step iv) is performed in an agent that improves the attachment of the blastocysts, and/or the inner cell mass cells to the feeder cells.

15. The method of claim 14, wherein the agent is a hyaluronic acid.

16. The method of claim 1, wherein the feeder cells are embryonic feeder cells.

17. The method of claim 1, wherein the feeder cells employed in steps ii) and iv) are the same or different and the feeder cells originate from animal source.

18. The method of claim 17, wherein the feeder cells are of mouse or human origin.

19. The method of claim 1, wherein the feeder cells are mitotically inactivated.

20. The method of claim 1, wherein the stem cell line
i) exhibits proliferation capacity in an undifferentiated state for more than 21 months when grown on mitotically inactivated embryonic feeder cells;
ii) exhibits normal euploid chromosomal karyotype;
iii) maintains potential to develop into derivatives of all types of germ layers both in vitro and in vivo;
iv) exhibits at least two of the group of molecular markers consisting of OCT-4, alkaline phosphatase, SSEA-3, SSEA-4, TRA 1-60, TRA 1-81, and the protein core of a keratin sulfate/chondroitin sulfate pericellular matrix proteinglycan recognized by the monoclonal antibody GCTM-2;
v) does not exhibit molecular marker SSEA-1 or other differentiation markers;
vi) retains pluripotency and forms teratomas in vivo when injected into immuno-compromised mice; and
vii) is capable of differentiation.

21. A blastocyst-derived stem cell line obtained by the method of claim 1.

22. The method of claim 1, wherein the stem cell line has the ability of differentiating into an insulin producing cells.

23. The method of claim 22, wherein the insulin producing cells form islet-like structures.

24. The method of claim 22, wherein the amount of insulin producing β-cells which is derived from the pluripotent human BS cell line is higher than 25%.

25. The method of claim 22, wherein the insulin producing cell line produces at least about 300 ng insulin/mg total protein.

26. The method of claim 1, wherein the blastocyst-derived stem cells have the ability to differentiate into differentiated cells, which display the expression of pancreatic cell type markers, including at least one of a group consisting of insulin, Glut-2, Pdx-1, glucokinase, glucagons, and somatostatin.

27. The method of claim 1, wherein the blastocyst-derived stem cells have the ability to differentiate into insulin-producing cells that organize into islet-like structures comprising an inner core of β-cells surrounded by an outer layer of neuron-type cells, which neuron-type cells display expression of at least one of the following neuronal cell type markers, including neuron-specific β-III tubulin (TUJ1), NeuN, DoubleCortin, tyrosine hydroxylase, and Map 2.

28. The method of claim 1, wherein the blastocyst-derived stem cells are capable of differentiated into cells, which express at least one neuronal cell type markers selected from the group consisting of, neuron-specific β-III tubulin (TUJ1), NeuN, DoubleCortin, tyrosine hydroxylase, and Map 2.

29. A preparation of differentiated cells derived from the blastocyst-derived stem cells obtained by the method of claim 1 for preventing or treating pathologies or diseases caused by tissue degeneration.

30. A preparation of differentiated cells derived from the blastocyst-derived stem cells obtained by the method of claim 1 for preventing or treating pathologies or diseases in the pancreas.

31. The preparation of differentiated cells of claim 30, wherein the disease is diabetes.

32. The preparation of differentiated cells of claim 28, wherein the disease is type 1 diabetes.

33. A preparation of differentiated cells derived from the blastocyst-derived stem cell line obtained by the method of claim 1 for preventing or treating pathologies or diseases in the nervous system.

34. The preparation of differentiated cells of claim 33, in which the disease is selected from the group consisting of multiple sclerosis, spinal chord injury, an encephalopathy, Parkinson's disease, Huntingdon's disease, stroke, a traumatic brain injury, a hypoxia induced brain injury, an ischemia induced brain injury, a hypoglycemic brain injury, a degenerative disorder of the nervous system, a brain tumor, and a neuropathy in the peripheral nervous system.

35. A kit for performing the method of claim 1, comprising human blastocysts with an intact zona pellucida or spontaneously hatched blastocysts, and at least two of the following components in separate compartments: hyaluronic acid, pronase, BS-cell medium, and human or mouse embryonic feeder cells.

36. A method for producing an essentially pure preparation of insulin-producing differentiated stem cells, comprising:
i) expanding human blastocyst-derived stem cells by growing the blastocyst-derived stem cells on an inactivated feeder cell layer in a suitable medium;
ii) generating blastocyst-derived stem cell bodies by dissociating colonies formed in step i) into smaller aggregates or individual cells, followed by transferring said aggregates or individual cells in to non-adherent containers wherein said aggregate or individual cells are incubated in a suitable medium;
iii) plating the blastocyst-derived stem cell bodies in containers in a suitable medium;
iv) selecting nestin-positive neural precursors in ITFSn medium;
v) expanding pancreatic endocrine progenitor cells in N2-medium comprising B27 media complement and basic fibroblast growth factor; and
vi) changing the medium to a basic fibroblast growth factor-free N2 medium.

37. The method of claim 36, wherein the human blastocyst-derived stem cells are obtained by:
i) using a fertilized oocyte of grade 1 or 2 to obtain a blastocyst of grade A or B:
ii) co-culturing the blastocyst with feeder cells to establish one or more colonies of inner cell mass cells,
iii) isolating the inner cell mass cells by mechanical dissection; and
iv) co-culturing the inner cell mass cells with feeder cells to obtain a blastocyst-derived stem cell line.

38. The method of claim 36, wherein the medium used in step i) is human blastocyst-derived stem cell medium.

39. The method of claim 36, wherein the medium used in step ii) is blastocyst-derived stem cell body medium.

40. The method of claim 36, wherein the medium used in step iii) is blastocyst-derived stem cell body medium.

41. The method of claim 36, wherein nicotinamide is added after step vi).

42. An essentially pure preparation of differentiated stem cells, wherein said stem cells display an expression of pancreatic cell type markers wherein said marker is at least one or more of insulin, Glut-2, Pdx-1, glucokinase, glucagons, or somatostatin.

43. The preparation of claim 42, which is capable of producing at least about 320 ng insulin/mg total protein.

44. The preparation of claim 42, wherein the preparation comprises at least 25% insulin producing cells.

45. The preparation of claim 42, wherein said stem cells are organized into islet-like structures comprising an inner core of β-cells surrounded by an outer layer of neuron-type cells, wherein the neuron-type cells express at least one of the neuronal cell type markers selected from the group consisting of: neuron-specific β-III tubulin (TUJ1), NeuN, DoubleCortin, tyrosine hydroxylase and Map 2.

46. The preparation of claim 42, obtained by:
i) expanding human blastocyst-derived stem cells by growing the blastocyst-derived stem cells on an inactivated feeder cell layer in a suitable medium;
ii) generating blastocyst-derived stem cell bodies by dissociating colonies formed in step i) into smaller aggregates or individual cells, followed by transferring said aggregates or individual cells in to non-adherent containers wherein said aggregate or individual cells are incubated in a suitable medium; and
iii) plating the blastocyst-derived stem cell bodies in containers in a suitable medium;
iv) selecting nestin-positive neural precursors in ITFSn medium;
v) expanding pancreatic endocrine progenitor cells in N2-medium comprising B27 media complement and basic fibroblast growth factor; and
vi) changing the medium to a basic fibroblast growth factor-free N2 medium.

47. An essentially pure preparation of differentiated stem cells, wherein the stem cells express at least one of the neuronal cell type markers selected from the group consisting of: neuron-specific β-III tubulin (TUJ1), NeuN, DoubleCortin, tyrosine hydroxylase, or Map 2.

48. The preparation of claim 47 obtained by:
i) expanding human blastocyst-derived stem cells by growing the blastocyst-derived stem cells on an inactivated feeder cell layer in a suitable medium;
ii) generating blastocyst-derived stem cell bodies by dissociating colonies formed in step i) into smaller aggregates or individual cells, followed by transferring said aggregates or individual cells in to non-adherent containers wherein said aggregate or individual cells are incubated in a suitable medium; and
iii) plating the blastocyst-derived stem cell bodies in containers in a suitable medium;
iv) selecting nestin-positive neural precursors in ITFSn medium;
v) expanding pancreatic endocrine progenitor cells in N2-medium comprising B27 media complement and basic fibroblast growth factor; and
vi) changing the medium to a basic fibroblast growth factor-free N2 medium.

49. An essentially pure preparation of stem cells obtained by:
i) expanding human blastocyst-derived stem cells by growing the blastocyst-derived stem cells on an inactivated feeder cell layer in a suitable medium;
ii) generating blastocyst-derived stem cell bodies by dissociating colonies formed in step i) into smaller aggregates or individual cells, followed by transferring said aggregates or individual cells in to non-adherent containers wherein said aggregate or individual cells are incubated in a suitable medium; and
iii) plating the blastocyst-derived stem cell bodies in containers in a suitable medium;
iv) selecting nestin-positive neural precursors in ITFSn medium,
v) expanding pancreatic endocrine progenitor cells in N2-medium comprising B27 media complement and basic fibroblast growth factor; and
vi) changing the medium to a basic fibroblast growth factor-free N2 medium.

50. An essentially pure preparation of differentiated stem cells of claim 42 for preventing or treating pathologies or diseases in the pancreas.

51. The preparation of claim 50, wherein the disease is diabetes.

52. The preparation of claim 50 wherein in which the disease is type 1 diabetes.

53. The preparation of claim 47 for treating pathologies or diseases in the nervous system.

54. The preparation of claim 53, wherein the disease is selected from the group consisting of multiple sclerosis, spinal chord injury, an encephalopathy, Parkinson's disease, Huntingdon's disease, stroke, a traumatic brain injury, a hypoxia induced brain injury, an ischemia induced brain injury, a hypoglycemic brain injury, a degenerative disorder of the nervous system, a brain tumor, and a neuropathy in the peripheral nervous system.

55. A kit for performing the method of claim 36 comprising at least two of the following components in separate compartments: mitomycin C, hBS medium, BS cell body medium, ITSFn-medium, N2-medium, B27-media supplement, nicotinamide, and bFGF.

56. The kit of claim 55, further comprising an essentially pure human blastocyst-derived stem cell line obtained by:
i) using a fertilized oocyte of grade 1 or 2 to obtain a blastocyst of grade A or B;
ii) co-culturing the blastocyst with feeder cells to establish one or more colonies of inner cell mass cells;
iii) isolating the inner cell mass cells by mechanical dissection; and
iv) co-culturing the inner cell mass cells with feeder cells to obtain a blastocyst-derived stem cell line.

57. The method of claim 1 further comprising propagating the blastocyst-derived stem cell line.

58. The method of claim 2 further comprising propagating the blastocyst-derived stem cell line.

59. The method of claim 3 further comprising propagating the blastocyst-derived stem cell line.

60. The method of claim 9, wherein the step of culturing uses feeder cells at a density less than about 55,000 cells per cm².

61. The method of claim 9, wherein the step of culturing uses feeder cells at a density less than about 50,000 cells per cm².
